(19) United States
(12) Patent Application Publication
(10) Pub. No.: US 2009/0304700 A1
(43) Pub. Date: Dec. 10, 2009

(54) CONFORMATION SPECIFIC ANTIBODIES THAT BIND TREFOIL FACTORS

(75) Inventor: PETER E. LOBIE, GLENDOWIE (NZ)

Correspondence Address: MINTZ, LEVIN, COHN, FERRIS, GLOVSKY AND POPEO, P.C, ONE FINANCIAL CENTER, BOSTON, MA, 02111, US

(73) Assignee: NEUREN PHARMACEUTICALS, LTD., NEWMARKET (NZ)

(21) Appl. No.: 12/478,912

(22) Filed: Jun. 5, 2009

Related U.S. Application Data
(60) Provisional application No. 61/059,558, filed on Jun. 6, 2008.

(51) Int. Cl. A61K 39/395; C07K 16/18; G01N 33/574; A61P 35/00

(52) U.S. Cl. 424/139.1; 530/387.9; 530/387.3; 435/7.23

(57) ABSTRACT

The present invention relates to conformation specific antibodies to TFF and compositions thereof. The present invention also relates to methods of regulation of cellular proliferation, survival and/or oncogenicity, particularly methods for the treatment of cancers, tumors and proliferative disorders.

### RELATED APPLICATIONS

This application claims the benefit of U.S. Provisional Application No. 61/059,558, filed Jun. 6, 2008, the contents of which are hereby incorporated by reference in their entirety.

### FIELD OF THE INVENTION

The invention relates generally to conformation specific antibodies to TFF and methods of using such antibodies to modulate, treat, prevent or delay the progression of cancer or a proliferative disorder.

### BACKGROUND OF THE INVENTION

The regulation and control of proliferation and/or survival of cells in animals is a complex process involving a number of cellular factors and their interactions with one another. Mutations or alteration in expression in any number of these cellular factors can result in uncontrolled proliferation or growth of cells and ultimately lead to the development of tumors and cancer.

Hormones and/or growth factors are involved in the normal regulation and control of cellular growth and development. For example, growth hormone (GH) is involved in normal pubertal mammary gland development (Walden et al., Endocrinology 139, 659-662, 1998; Kleinberg, J. Mammary Gland Biol. Neoplasia. 2, 49-57, 1997; Bchini et al., (1991) Endocrinology 128, 539-546, 1991; Tornell et al., Int. J. Cancer 49, 114-11, 1991; Nagasawa et al., Eur. J. Cancer Clin. Oncol. 21, 1547-1551, 1985; Swanson and Unterman, Carcinogenesis 23, 977-982, 2002; Stavrou and Kleinberg, Endocrinol. Metab. Clin. North Am. 30, 545-563, 2001; Okada and Kopchick, Trends Mol. Med 7, 126-132, 2001; Ng et al., Nat. Med 3, 1141-1144, 1997) and expressed in normal human mammary gland (Raccurt et al., J. Endocrinol. 175, 307-318, 2002). Alterations in expression levels of hormones such as GH can result in aberrant proliferation of cells. For example, increased epithelial expression of the hGH gene is associated with the acquisition of pathological proliferation, and the highest level of hGH gene expression is observed in metastatic mammary carcinoma cells (Raccurt et al., J. Endocrinol. 175, 307-318, 2002). Such alterations in expression of autocrine hGH may result in the transformation of a normal cell to a cancer cell.

There is a need to understand further the effects of hormones and/or growth factors on the development of proliferative disorders, including identifying any cellular factors which promote cell proliferation, cell survival and/or oncogenic transformation. This will aid in the identification of means for the regulation of proliferation and/or survival, and particularly means for the treatment of proliferative disorders such as cancer.

### SUMMARY OF THE INVENTION

The invention provides conformation specific antibodies to TFF and methods of using such antibodies to modulate, e.g., reduce, inhibit, treat or prevent cancer or a proliferative disorder, and/or to modulate, e.g., reduce, inhibit, or delay the progression of a cancer or proliferative disorder.

The conformation specific antibodies to TFF described herein include antibodies that specifically bind a trefoil factor 1 (TFF1) polypeptide, wherein the antibody binds to a conformational epitope on the TFF1 polypeptide. For example, the conformational epitope is selected from a conformational epitope shown in Table 3.

The conformation specific antibodies to TFF described herein include antibodies that specifically bind a trefoil factor 1 (TFF1) polypeptide, wherein the antibody immunoreacts with an antigenic determinant selected from the antigenic determinants shown in Table 4.

The conformation specific antibodies to TFF described herein include antibodies that specifically bind a trefoil factor 1 (TFF1) homodimer polypeptide, wherein the antibody binds to a conformational epitope on the TFF1 polypeptide. For example, the conformational epitope is selected from a conformational epitope shown in Table 1.

The conformation specific antibodies to TFF described herein include antibodies that specifically bind a trefoil factor 1 (TFF1) monomer, homodimer or heterodimer polypeptide, wherein the antibody immunoreacts with an antigenic determinant shown in Table 2.

The conformation specific antibodies to TFF described herein include antibodies produced by hybridoma cell lines and referred to herein as 2B10 and 1F9.

The conformation specific antibodies to TFF provided herein specifically bind to a trefoil factor polypeptide. For example, in some embodiments, the conformation specific antibodies to TFF bind to TFF1. In some embodiments, the conformation specific antibodies to TFF bind to TFF3. The invention also provides multivalent antibodies that recognize both TFF1 and TFF3. These antibodies are also referred to herein as multimeric or bispecific antibodies. For example, in some embodiments, the TFF specific antibody is a heterodimer. In some embodiments, the TFF specific antibody is a chimeric antibody in which the antigen-binding fragment of the antibody from one species is fused with constant region from another species. For example, the TFF specific antibody is a humanized antibody. In some embodiments, the chimeric antibody compositions contain a TFF1 binding component. In some embodiments, the chimeric antibody compositions contain a TFF1 binding component and a TFF3 binding component.

The term “antibody” is used herein in the broadest sense and is intended to include intact monoclonal antibodies and polyclonal antibodies, as well as derivatives, variants, fragments and/or any other modification thereof so long as they exhibit the desired biological activity. Antibodies encompass immunoglobulin molecules and immunologically active portions of immunoglobulin (Ig) molecules, i.e., molecules that contain an antigen binding site that specifically binds (immunoreacts with) an antigen. These include, but are not limited to, polyclonal, monoclonal, chimeric, single chain, Fc, Fab, Fab′, and Fab₂ fragments, and a Fab expression library. Antibody molecules relate to any of the classes IgG, IgM, IgA, IgE, and IgD, which differ from one another by the nature of heavy chain present in the molecule. These include subclasses as well, such as IgG1, IgG2, and others. The light chain may be a kappa chain or a lambda chain. Reference herein to antibodies includes a reference to all classes, subclasses, and types. Also included are chimeric antibodies, for example, monoclonal antibodies or modifications thereof that are specific to more than one source, e.g., a mouse or human sequence. Further included are camelid antibodies or nanobodies. TFF binding antibodies also include multi-specific, e.g., bispecific (e.g., multivalent, or multimeric) antibodies and functional fragments thereof. The terms “TFF binding antibodies” and “TFF antibodies” are used interchangeably herein. It will be understood that each reference to “antibodies” or any like term, herein includes intact antibodies, as well as any modifications thereof.

Conformation specific antibodies to TFF include those that bind to domains or residues that are exposed, e.g., outer loop structure residues in the tertiary structure of the protein in solution, participate in TFF dimerization, aggregation, as well as domains responsible for promoting cellular proliferation, survival, and oncogenicity. For example, the epitope binding specificity of the antibody includes a TFF sequence that contains a domain involved in stimulation of cell proliferation, survival, oncogenicity and migration/invasion. For example, an antibody binds to a conformational epitope provided herein in Tables 1, 3, or 5. The antibody is a polyclonal antisera or monoclonal antibody or derivative of either of those. The invention encompasses not only an intact monoclonal antibody, but also an immunologically-active antibody fragment, e.g., a Fab or (Fab)₂ fragment; an engineered single chain Fv molecule; or a chimeric molecule, e.g., an antibody which contains the binding specificity of one antibody, e.g., of murine origin, and the remaining portions of another antibody, e.g., of human origin.

TFF binding antibodies are used to modulate, e.g., reduce or otherwise inhibit, completely or partially, the ability of the target TFF molecule (i.e., the TFF antigen to which a given TFF binding antibody binds) to bind or otherwise interact with another TFF molecule. The additional TFF molecule can be the same as the target TFF molecule, as in the case of homomultimerization, e.g., homodimerization, or the additional TFF molecule can differ from the target TFF molecule.

TFF binding antibodies are also used to modulate, e.g., reduce or otherwise inhibit, completely or partially, the ability of the target TFF molecule to bind or otherwise interact with a second molecule, such as, for example, a cognate TFF receptor molecule, an extracellular receptor or other cell-surface and/or intracellular signaling molecules.

Other TFF-binding antibodies are used to directly target TFF-over-expressing cells for destruction. In the latter case, the antibody, or fragment thereof, activates complement in a patient treated with the antibody. In some instances, the antibody mediates antibody-dependent cytotoxicity of tumor cells in the patient treated with the antibody. The antibody, or fragment thereof, is administered alone or conjugated to a cytotoxic agent. Binding of the antibody to a tumor cell expressing TFF, or a TFF binding molecule, results in impairment or death of the cell, thereby reducing tumor load. The antibody is optionally conjugated to a radiochemical, or a chemical tag which sensitizes the cell to which it is bound to radiation or laser-mediated killing.

Optionally, the antibody compositions contain a pharmaceutically acceptable carrier and/or a second compound. For example, the second compound is a chemotherapeutic or anti-neoplastic agent. Such agents are administered sequentially, e.g., prior to, or after the administration of the TFF antibody, or simultaneously, e.g., co-administration or co-therapy.

The conformation specific antibodies to TFF described herein are used in methods of inhibiting proliferation, survival, and/or oncogenicity of a tumor cell by contacting the cell, a biological sample suspected of containing a tumor cell, an extracellular receptor, such as a TFF receptor, or another cell-surface protein on the tumor cell with any of the conformation specific antibodies to TFF described herein, or with combinations of these antibodies.

The conformation specific antibodies to TFF described herein are used in methods of preventing cancer or a cell proliferation and/or survival disorder in a subject in need thereof by administering any of the conformation specific antibodies to TFF described herein, or by administering combinations of these antibodies.

The conformation specific antibodies to TFF described herein are used to treat a cancer or tumor. For example, the tumor or cancer is an epithelial tumor such as, e.g., lung cancer, colorectal cancer, breast cancer, pancreatic cancer, ovarian cancer, prostate cancer, hepatic carcinoma, gastric carcinoma, endometrial carcinoma, renal carcinoma, thyroid cancer, biliary duct cancer, esophageal cancer, brain cancer, melanoma, multiple myeloma, hematologic tumor, and lymphoid tumor.

The conformation specific antibodies to TFF described herein are used to treat a proliferative disorder. Proliferative disorders include, e.g., keratinocyte hyperproliferation, inflammatory cell infiltration, cytokine alteration, endometriosis, epidermic and dermoid cysts, lipomas, adenomas, capillary and cutaneous hemangiomas, lymphangiomas, nevi lesions, teratomas, nephromas, myofibromatosis, osteoplastic tumors, and other dysplastic masses.

The subject is a mammal, preferably a human suffering from or at risk of developing a tumor or cancer or proliferative disorder. The compositions and methods are also useful for veterinary use, e.g., in treating, cats, dogs, and other pets in addition to livestock, horses, cattle and the like.

The conformation specific antibodies to TFF described herein are also useful in a variety of diagnostic applications. The invention features a method for diagnosing cancer or a cell proliferation and/or survival disorder in a mammal by contacting a tissue or bodily fluid from the mammal with a conformation specific antibody to TFF under conditions sufficient to form an antigen-antibody complex and detecting the antigen-antibody complex. Cancers or tumors detected using the conformation specific antibodies to TFF described herein include an epithelial tumor such as, e.g., lung cancer, colorectal cancer, breast cancer, pancreatic cancer, ovarian cancer, prostate cancer, hepatic carcinoma, gastric carcinoma, endometrial carcinoma, renal carcinoma, thyroid cancer, biliary duct cancer, esophageal cancer, brain cancer, melanoma, multiple myeloma, hematologic tumor, and lymphoid tumor. Proliferative disorders detected using the conformation specific antibodies to TFF described herein include, e.g., keratinocyte hyperproliferation, inflammatory cell infiltration, cytokine alteration, endometriosis, epidermic and dermoid cysts, lipomas, adenomas, capillary and cutaneous hemangiomas, lymphangiomas, nevi lesions, teratomas, nephromas, myofibromatosis, osteoplastic tumors, and other dysplastic masses.

Patient derived tissue samples, e.g., biopsies of solid tumors, as well as bodily fluids such as a CNS-derived bodily fluid, blood, serum, urine, saliva, sputum, lung effusion, and ascites fluid, are contacted with conformation specific antibodies to TFF.

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described below. All publications, patent applications, patents, and other references mentioned herein are incorporated by reference. In the case of conflict, the present specification, including definitions, will control. In addition, the materials, methods, and examples are illustrative only and are not intended to be limiting.

Other features and advantages of the invention will be apparent from the following detailed description and claims.

### BRIEF DESCRIPTION OF THE DRAWINGS

FIG. 1 is a graph illustrating the effect of mouse monoclonal TFF1 antibodies on the viability of TFF1 positive AGS human gastric carcinoma cells in vitro in comparison to control mouse IgG (IgG).

FIG. 2A is a graph illustrating that mouse monoclonal TFF1 antibody 2B10 reduced the viability of TFF1 positive AGS human gastric carcinoma cells in vitro in a dose dependent manner in comparison to control mouse IgG (mIgG). FIG. 2B includes representative photomicrographs of the gastric carcinoma cells after 72 hours incubation with TFF1 monoclonal antibody 2B10 compared to control IgG (left-side, low magnification; right side, high magnification).

FIG. 3A is a graph illustrating that mouse monoclonal TFF1 antibody 2B10 reduced the viability of TFF1 positive MCF-7 mammary carcinoma cells in vitro in a dose dependent manner in comparison to control mouse IgG (mIgG). FIG. 3B includes representative photomicrographs of the mammary carcinoma cell after 72 hours incubation with TFF1 monoclonal antibody 2B10 compared to control IgG.

FIG. 4 is a graph illustrating that mouse monoclonal TFF1 antibody 2B10 reduced the viability of TFF1 positive T47-D human mammary carcinoma cells in vitro in comparison to control mouse IgG (mIgG).

FIG. 5 is a graph illustrating that mouse monoclonal TFF1 antibody 2B10 did not reduce the viability of TFF1 negative MDA-MB-231 human mammary carcinoma cells in vitro in comparison to control mouse IgG (mIgG).

FIG. 6 is a graph illustrating that mouse monoclonal TFF1 antibody 1F9 reduced the viability of TFF1 positive human mammary carcinoma cells (T47D and MCF7) and human gastric carcinoma cells (AGS) in vitro in comparison to control mouse IgG (mIgG), but did not reduce the viability of TFF1 negative human mammary epithelial (MCF10A) cells in vitro.

### DETAILED DESCRIPTION OF THE INVENTION

The trefoil factor family of proteins are characterized by a 40-amino acid trefoil motif that contains 3 conserved disulfide bonds. The 3 intrachain disulfide bonds form the trefoil motif (TFF domain). The trefoil motif is known in the art, e.g. Taupin and Podolsky, Nat Rev Mol Cell Bio. 4(9):721-32, 2003; Hoffmann et al., Histol Histopathol 16(1):319-34, 2001; and Thim, Cell Mol Life Sci 53(11-12):888-903, 1997.

In humans, three distinct members of the trefoil peptides have been identified. TFF1 or pS2 was first detected in a mammary cancer cell line as an estrogen-inducible gene. In human stomach, it is predominantly located in the foveolar cells of the gastric mucosa. TFF2 (formerly spasmolytic polypeptide or SP) was first purified from porcine pancreas and is expressed in mucous neck cells, deep pyloric glands, and Brunner's glands. TFF3 or intestinal trefoil factor (ITF) was the last to be identified and is predominantly expressed in the goblet cells of the small and large intestine. The trefoil peptides are involved in mucosal healing processes and are expressed at abnormal elevated levels in neoplastic diseases. A wide range of human carcinomas and gastrointestinal inflammatory malignancies, including peptic ulceration and colitis, Crohn's syndrome, pancreatitis, and biliary disease, aberrantly express trefoil peptides. Additionally, TFF protein expression has been confirmed in various human cancer cell lines using RT-PCR analysis. For example, RT-PCR analysis has confirmed TFF1 and TFF3 expression in prostate (PC3 and LNCaP), mammary (MCF-7 and T47-D), gastric (AGS), and colorectal (DLD-1 and HT-29) carcinoma cells. Orthologues of the human TFF proteins have been identified in other animals; for example, rats, mice and primates.

The trefoil family of peptides possess divergent functions in the mammary gland with TFF1 functioning as a mitogen and TFF2 stimulating branching morphogenesis and cell survival. TFF3 is widely co-expressed with TFF1 in malignancies of the human mammary gland whereas TFF2 is not expressed in the mammary epithelial cells.

Reference herein to “TFF”, “TFF protein(s)”, or “TFF family of proteins” refers to the group of related proteins including TFF1, TFF2, and TFF3. TFF proteins share at least approximately 28 to 45% amino acid identity within the same species.

Recombinant human TFF proteins (e.g., TFF1 and TFF3) have been produced using a Glutathione S-transferase (GST) gene fusion system to produce the recombinant proteins in E. coli bacteria, and purified for use as antigens for antibody production, as described in U.S. Ser. No. 11/906968 filed on Oct. 3, 2007 (Published as US 20080199455) and PCT/US2007/021355 filed on Oct. 3, 2007 (Published as WO2008/042435), each of which are hereby incorporated by reference in their entirety. Native-PAGE and western blot analysis demonstrated that recombinant native TFF1 principally resolves as a monomer, and to a lesser extent, a dimer, whereas recombinant TFF3 resolves as a multimeric form. Conformational antibodies specific for TFF1 and TFF3, respectively, have been identified using these purified, native TFF proteins. The antibodies described in US 20080199455 and WO2008/042435 were shown to be highly sensitive and specific against the antigens against which they were raised (e.g., TFF1 or TFF3), with no cross reactivity against other trefoil factor proteins. The antibodies described therein were also shown to be highly functional against a variety of human carcinoma cells, and were shown to induce apoptosis and reduce cell viability of a variety of human carcinoma cell lines.

The present invention provides novel, conformation specific TFF antibodies, e.g., monoclonal TFF1 antibodies. Exemplary antibodies of the invention include, for example, the monoclonal TFF1 antibody referred to herein as “2B10”, and the monoclonal TFF1 antibody referred to herein as “1F9”. These antibodies show specificity for TFF1 and they have been shown to modulate, e.g., reduce or otherwise inhibit, the viability of human carcinoma cell lines which express TFF1.

The present invention also provides conformation specific antibodies having the same or similar specificity as a TFF antibody described herein. Those skilled in the art will recognize that it is possible to determine, without undue experimentation, if an antibody has the same specificity as a TFF antibody described herein by ascertaining whether the former prevents the latter from binding to a CD3 antigen polypeptide. If the antibody being tested competes with an antibody of the invention, as shown by a decrease in binding by the TFF antibody of the invention, then the two antibodies bind to the same, or a closely related, epitope. Another way to determine whether an antibody has the specificity of an antibody of the invention is to pre-incubate the antibody of the invention with the TFF antigen with which it is normally reactive, i.e., TFF1, and then add the antibody being tested to determine if the antibody being tested is inhibited in its ability to bind the TFF antigen. If the antibody being tested is inhibited then, it is likely to have the same, or functionally equivalent, epitopic specificity as the antibody of the invention.

Various procedures known within the art may be used for the production of polyclonal or monoclonal antibodies directed against TFF proteins, or against derivatives, fragments, analogs homologs or orthologs thereof. (See, for example, Antibodies: A Laboratory Manual, Harlow E, and Lane D, 1988, Cold Spring Harbor Laboratory Press, Cold Spring Harbor, N.Y., incorporated herein by reference).

Antibodies are purified by well-known techniques, such as affinity chromatography using protein A or protein G, which provide primarily the IgG fraction of immune serum. Subsequently, or alternatively, the specific antigen which is the target of the immunoglobulin sought, or an epitope thereof, may be immobilized on a column to purify the immune specific antibody by immunoaffinity chromatography. Purification of immunoglobulins is discussed, for example, by D. Wilkinson (The Scientist, published by The Scientist, Inc., Philadelphia Pa., Vol. 14, No. 8 (Apr. 17, 2000), pp. 25-28).

As used herein, the term “antibody” refers to immunoglobulin molecules and immunologically active portions of immunoglobulin (Ig) molecules, i.e., molecules that contain an antigen binding site that specifically binds (immunoreacts with) an antigen. Such antibodies include, but are not limited to, polyclonal, monoclonal, chimeric, single chain, F_{ab}, F_{ab′} and F_{(ab′)2} fragments, and an F_{ab} expression library. By “specifically bind” or “immunoreacts with” is meant that the antibody reacts with one or more antigenic determinants of the desired antigen and does not react (i.e., bind) with other polypeptides or binds at much lower affinity (K_{d}>10⁻⁶) with other polypeptides.

The basic antibody structural unit is known to comprise a tetramer. Each tetramer is composed of two identical pairs of polypeptide chains, each pair having one “light” (about 25 kDa) and one “heavy” chain (about 50-70 kDa). The amino-terminal portion of each chain includes a variable region of about 100 to 110 or more amino acids primarily responsible for antigen recognition. The carboxy-terminal portion of each chain defines a constant region primarily responsible for effector function. Human light chains are classified as kappa and lambda light chains. Heavy chains are classified as mu, delta, gamma, alpha, or epsilon, and define the antibody's isotype as IgM, IgD, IgA, and IgE, respectively. Within light and heavy chains, the variable and constant regions are joined by a “J” region of about 12 or more amino acids, with the heavy chain also including a “D” region of about 10 more amino acids. See generally, Fundamental Immunology Ch. 7 (Paul, W., ea., 2nd ed. Raven Press, N.Y. (1989)). The variable regions of each light/heavy chain pair form the antibody binding site.

The term “monoclonal antibody” (MAb) or “monoclonal antibody composition”, as used herein, refers to a population of antibody molecules that contain only one molecular species of antibody molecule consisting of a unique light chain gene product and a unique heavy chain gene product. In particular, the complementarity determining regions (CDRs) of the monoclonal antibody are identical in all the molecules of the population. MAbs contain an antigen binding site capable of immunoreacting with a particular epitope of the antigen characterized by a unique binding affinity for it.

In general, antibody molecules obtained from humans relate to any of the classes IgG, IgM, IgA, IgE and IgD, which differ from one another by the nature of the heavy chain present in the molecule. Certain classes have subclasses as well, such as IgG₁, IgG₂, and others. Furthermore, in humans, the light chain may be a kappa chain or a lambda chain.

The term “antigen-binding site” or “binding portion” refers to the part of the immunoglobulin molecule that participates in antigen binding. The antigen binding site is formed by amino acid residues of the N-terminal variable (“V”) regions of the heavy (“H”) and light (“L”) chains. Three highly divergent stretches within the V regions of the heavy and light chains, referred to as “hypervariable regions,” are interposed between more conserved flanking stretches known as “framework regions,” or “FRs”. Thus, the term “FR” refers to amino acid sequences which are naturally found between, and adjacent to, hypervariable regions in immunoglobulins. In an antibody molecule, the three hypervariable regions of a light chain and the three hypervariable regions of a heavy chain are disposed relative to each other in three dimensional space to form an antigen-binding surface. The antigen-binding surface is complementary to the three-dimensional surface of a bound antigen, and the three hypervariable regions of each of the heavy and light chains are referred to as “complementarity-determining regions,” or “CDRs.” The assignment of amino acids to each domain is in accordance with the definitions of Kabat Sequences of Proteins of Immunological Interest (National Institutes of Health, Bethesda, Md. (1987 and 1991)), or Chothia & Lesk J. Mol. Biol. 196:901-917 (1987), Chothia et al. Nature 342:878-883 (1989).

As used herein, the term “epitope” or “antigenic determinant” includes any accessible feature of an antigen capable of specific binding to an immunoglobulin, an scFv, or a T-cell receptor. The term “epitope” includes any protein determinant capable of specific binding to an immunoglobulin or T-cell receptor. Epitopic determinants usually consist of chemically active surface groupings of molecules such as amino acids or sugar side chains and usually have specific three dimensional structural characteristics, as well as specific charge characteristics. For example, the antigenic determinant or surface feature of an antigen that immunoreacts with a conformational specific antigen contains amino acid residues of the antigen that are not necessarily contiguous with respect to the linear amino acid sequence of the protein. An antibody is said to specifically bind an antigen when the dissociation constant is ≦1 μM; preferably ≦100 nM and most preferably ≦10 nM.

Monoclonal antibodies that modulate, block, inhibit, reduce, antagonize, neutralize or otherwise interfere with the bioactivity of one or more TFF proteins (e.g., TFF1) are generated, e.g., by immunizing an animal with recombinant TFF protein, such as, for example, murine, rat or human recombinant TFF1 protein, or an immunogenic fragment, derivative or variant thereof. Alternatively, the animal is immunized with cells transfected with a vector containing a nucleic acid molecule encoding TFF protein, such that TFF protein is expressed and associated with the surface of the transfected cells. Alternatively, the antibodies are obtained by screening a library that contains antibody or antigen binding domain sequences for binding to TFF protein. This library is prepared, e.g., in bacteriophage as protein or peptide fusions to a bacteriophage coat protein that is expressed on the surface of assembled phage particles and the encoding DNA sequences contained within the phage particles (i.e., “phage displayed library”). Hybridomas resulting from myeloma/B cell fusions are then screened for reactivity to one or more TFF proteins (e.g., TFF1, TFF2 and/or TFF3).

Monoclonal antibodies are prepared, for example, using hybridoma methods, such as those described by Kohler and Milstein, Nature, 256:495 (1975). In a hybridoma method, a mouse, hamster, or other appropriate host animal, is typically immunized with an immunizing agent to elicit lymphocytes that produce or are capable of producing antibodies that will specifically bind to the immunizing agent. Alternatively, the lymphocytes can be immunized in vitro.

The immunizing agent will typically include the protein antigen, a fragment thereof or a fusion protein thereof. Generally, either peripheral blood lymphocytes are used if cells of human origin are desired, or spleen cells or lymph node cells are used if non-human mammalian sources are desired. The lymphocytes are then fused with an immortalized cell line using a suitable fusing agent, such as polyethylene glycol, to form a hybridoma cell (Goding, Monoclonal Antibodies: Principles and Practice, Academic Press, (1986) pp. 59-103). Immortalized cell lines are usually transformed mammalian cells, particularly myeloma cells of rodent, bovine and human origin. Usually, rat or mouse myeloma cell lines are employed. The hybridoma cells can be cultured in a suitable culture medium that preferably contains one or more substances that inhibit the growth or survival of the unfused, immortalized cells. For example, if the parental cells lack the enzyme hypoxanthine guanine phosphoribosyl transferase (HGPRT or HPRT), the culture medium for the hybridomas typically will include hypoxanthine, aminopterin, and thymidine (“HAT medium”), which substances prevent the growth of HGPRT-deficient cells.

Preferred immortalized cell lines are those that fuse efficiently, support stable high level expression of antibody by the selected antibody-producing cells, and are sensitive to a medium such as HAT medium. More preferred immortalized cell lines are murine myeloma lines, which can be obtained, for instance, from the Salk Institute Cell Distribution Center, San Diego, Calif. and the American Type Culture Collection, Manassas, Va. Human myeloma and mouse-human heteromyeloma cell lines also have been described for the production of monoclonal antibodies. (See Kozbor, J. Immunol., 133:3001 (1984); Brodeur et al., Monoclonal Antibody Production Techniques and Applications, Marcel Dekker, Inc., New York, (1987) pp. 51-63)).

The culture medium in which the hybridoma cells are cultured can then be assayed for the presence of monoclonal antibodies directed against the antigen. Preferably, the binding specificity of monoclonal antibodies produced by the hybridoma cells is determined by immunoprecipitation or by an in vitro binding assay, such as radioimmunoassay (RIA) or enzyme-linked immunoabsorbent assay (ELISA). Such techniques and assays are known in the art. The binding affinity of the monoclonal antibody can, for example, be determined by the Scatchard analysis of Munson and Pollard, Anal. Biochem., 107:220 (1980). Moreover, in therapeutic applications of monoclonal antibodies, it is important to identify antibodies having a high degree of specificity and a high binding affinity for the target antigen.

After the desired hybridoma cells are identified, the clones can be subcloned by limiting dilution procedures and grown by standard methods. (See Goding, Monoclonal Antibodies: Principles and Practice, Academic Press, (1986) pp. 59-103). Suitable culture media for this purpose include, for example, Dulbecco's Modified Eagle's Medium and RPMI-1640 medium. Alternatively, the hybridoma cells can be grown in vivo as ascites in a mammal.

The monoclonal antibodies secreted by the subclones can be isolated or purified from the culture medium or ascites fluid by conventional immunoglobulin purification procedures such as, for example, protein A-Sepharose, hydroxylapatite chromatography, gel electrophoresis, dialysis, or affinity chromatography.

Monoclonal antibodies can also be made by recombinant methods, such as those described in U.S. Pat. No. 4,816,567. Polynucleotides, e.g., DNA encoding the monoclonal antibodies of the invention can be readily isolated and sequenced using conventional procedures (e.g., by using oligonucleotide probes that are capable of binding specifically to genes encoding the heavy and light chains of murine antibodies). The term “isolated polynucleotide” as used herein shall mean a polynucleotide of genomic, cDNA, or synthetic origin or some combination thereof, which by virtue of its origin the “isolated polynucleotide” (1) is not associated with all or a portion of a polynucleotide in which the “isolated polynucleotide” is found in nature, (2) is operably linked to a polynucleotide which it is not linked to in nature, or (3) does not occur in nature as part of a larger sequence. The terms “isolated polypeptide” or “isolated protein” referred to herein means a protein of cDNA, recombinant RNA, or synthetic origin or some combination thereof, which by virtue of its origin, or source of derivation, the “isolated protein” (1) is not associated with proteins found in nature, (2) is free of other proteins from the same source, e.g., free of marine proteins, (3) is expressed by a cell from a different species, or (4) does not occur in nature.

The hybridoma cells of the invention serve as a preferred source of such DNA. Once isolated, the DNA can be placed into expression vectors, which are then transfected into host cells such as simian COS cells, Chinese hamster ovary (CHO) cells, or myeloma cells that do not otherwise produce immunoglobulin protein, to obtain the synthesis of monoclonal antibodies in the recombinant host cells. The DNA also can be modified, for example, by substituting the coding sequence for human heavy and light chain constant domains in place of the homologous murine sequences (see U.S. Pat. No. 4,816,567; Morrison, Nature 368, 812-13 (1994)) or by covalently joining to the immunoglobulin coding sequence all or part of the coding sequence for a non-immunoglobulin polypeptide. Such a non-immunoglobulin polypeptide can be substituted for the constant domains of an antibody of the invention, or can be substituted for the variable domains of one antigen-combining site of an antibody of the invention to create a chimeric bivalent antibody.

Monoclonal antibodies of the invention include humanized antibodies or human antibodies. These antibodies are suitable for administration to humans without engendering an immune response by the human against the administered immunoglobulin. Humanized forms of antibodies are chimeric immunoglobulins, immunoglobulin chains or fragments thereof (such as Fv, Fab, Fab′, F(ab′)₂ or other antigen-binding subsequences of antibodies) that are principally comprised of the sequence of a human immunoglobulin, and contain minimal sequence derived from a non-human immunoglobulin. Humanization is performed, e.g., by following the method of Winter and co-workers (Jones et al., Nature, 321:522-525 (1986); Riechmann et al., Nature, 332:323-327 (1988); Verhoeyen et al., Science, 239:1534-1536 (1988)), by substituting rodent CDRs or CDR sequences for the corresponding sequences of a human antibody. (See also U.S. Pat. No. 5,225,539). In some instances, Fv framework residues of the human immunoglobulin are replaced by corresponding non-human residues. Humanized antibodies also comprise, e.g., residues which are found neither in the recipient antibody nor in the imported CDR or framework sequences. In general, the humanized antibody includes substantially all of at least one, and typically two, variable domains, in which all or substantially all of the CDR regions correspond to those of a non-human immunoglobulin and all or substantially all of the framework regions are those of a human immunoglobulin consensus sequence. The humanized antibody optimally also includes at least a portion of an immunoglobulin constant region (Fc), typically that of a human immunoglobulin (Jones et al., 1986; Riechmann et al., 1988; and Presta, Curr. Op. Struct. Biol., 2:593-596 (1992)).

Fully human antibodies are antibody molecules in which the entire sequence of both the light chain and the heavy chain, including the CDRs, arise from human genes. Such antibodies are termed “human antibodies” or “fully human antibodies” herein. Monoclonal antibodies can be prepared by using trioma technique; the human B-cell hybridoma technique (see Kozbor, et al., 1983 Immunol Today 4: 72); and the EBV hybridoma technique to produce monoclonal antibodies (see Cole, et al., 1985 In: MONOCLONAL ANTIBODIES AND CANCER THERAPY, Alan R. Liss, Inc., pp. 77-96). Monoclonal antibodies may be utilized and may be produced by using human hybridomas (see Cote, et al., 1983. Proc Natl Acad Sci USA 80: 2026-2030) or by transforming human B-cells with Epstein Barr Virus in vitro (see Cole, et al., 1985 In: MONOCLONAL ANTIBODIES AND CANCER THERAPY, Alan R. Liss, Inc., pp. 77-96).

Human antibodies may additionally be produced using transgenic nonhuman animals which are modified so as to produce fully human antibodies rather than the animal's endogenous antibodies in response to challenge by an antigen. (See PCT publication WO94/02602). The endogenous genes encoding the heavy and light immunoglobulin chains in the nonhuman host have been incapacitated, and active loci encoding human heavy and light chain immunoglobulins are inserted into the host's genome. The human genes are incorporated, for example, using yeast artificial chromosomes containing the requisite human DNA segments. An animal which provides all the desired modifications is then obtained as progeny by crossbreeding intermediate transgenic animals containing fewer than the full complement of the modifications. An example of such a nonhuman animal is a mouse termed the Xenomouse™ as disclosed in PCT publications WO 96/33735 and WO 96/34096. See also U.S. Pat. No. 5,939,598 and U.S. Pat. No. 5,916,771 for additional examples of methods for producing humanized antibodies.

The antibody can be expressed by a vector containing a DNA segment encoding the single chain antibody described above. These can include vectors, liposomes, naked DNA, adjuvant-assisted DNA. gene gun, catheters, etc. The vectors can be chromosomal, non-chromosomal or synthetic. Preferred vectors include viral vectors, fusion proteins and chemical conjugates. The particular vector chosen will depend upon the target cell and the condition being treated. The introduction can be by standard techniques, e.g. infection, transfection, transduction or transformation. Examples of modes of gene transfer include e.g., naked DNA, CaPO₄ precipitation, DEAE dextran, electroporation, protoplast fusion, lipofection, cell microinjection, and viral vectors.

The vector can be employed to target essentially any desired target cell using catheters, intravenous, parenteral, intraperitoneal and subcutaneous injection, and oral or other known routes of administration. These vectors can be used to express large quantities of antibodies that can be used in a variety of ways. For example, to detect the presence of a TFF protein (e.g., TFF1, TFF2 and/or TFF3) in a sample. The antibody can also be used to try to bind to and disrupt the bioactivity of one or more TFF proteins (e.g., TFF1, TFF2 and/or TFF3).

The invention also includes and bispecific TFF antibodies. Bispecific antibodies are antibodies that have binding specificities for at least two different antigens. In the present case, one of the binding specificities is for a TFF protein (e.g., TFF1, TFF2 or TFF3). The second binding target is any other antigen, e.g., a different TFF protein, a cell-surface protein or receptor or receptor subunit. These antibodies are also referred to herein as multivalent and multimeric antibodies.

Methods for making bispecific antibodies are known in the art. Traditionally, the recombinant production of bispecific antibodies is based on the co-expression of two immunoglobulin heavy-chain/light-chain pairs, where the two heavy chains have different specificities (Milstein and Cuello, Nature, 305:537-539 (1983)). For further details of generating bispecific antibodies see, for example, Suresh et al., Methods in Enzymology, 121:210 (1986). See also WO 96/27011.

Antibodies with more than two valencies are contemplated. For example, trispecific antibodies can be prepared. Tutt et al., J. Immunol. 147:60 (1991).

Heteroconjugate antibodies are also within the scope of the present invention. Heteroconjugate antibodies are composed of two covalently joined antibodies. It is contemplated that the antibodies can be prepared in vitro using known methods in synthetic protein chemistry, including those involving crosslinking agents.

The invention also pertains to immunoconjugates comprising an antibody conjugated to a cytotoxic agent such as a toxin (e.g., an enzymatically active toxin of bacterial, fungal, plant, or animal origin, or fragments thereof), or a radioactive isotope (i.e., a radioconjugate). Conjugates of the antibody and cytotoxic agent are made using a variety of bifunctional protein-coupling agents. For example, carbon-14-labeled 1-isothiocyanatobenzyl-3-methyldiethylene triaminepentaacetic acid (MX-DTPA) is an exemplary chelating agent for conjugation of radionucleotide to the antibody. (See WO94/11026).

Those of ordinary skill in the art will recognize that a large variety of possible moieties can be coupled to the resultant antibodies of the invention. (See, for example, “Conjugate Vaccines”, Contributions to Microbiology and Immunology, J. M. Cruse and R. E. Lewis, Jr (eds), Carger Press, New York, (1989), the entire contents of which are incorporated herein by reference). Coupling may be accomplished by any chemical reaction that will bind the two molecules so long as the antibody and the other moiety retain their respective activities. This linkage can include many chemical mechanisms, for instance covalent binding, affinity binding, intercalation, coordinate binding and complexation.

The antibodies disclosed herein can also be formulated as immunoliposomes. Liposomes containing the antibody are prepared by methods known in the art, such as described in Epstein et al., Proc. Natl. Acad. Sci. USA, 82: 3688 (1985); Hwang et al., Proc. Natl Acad. Sci. USA, 77: 4030 (1980); and U.S. Pat. Nos. 4,485,045 and 4,544,545. Liposomes with enhanced circulation time are disclosed in U.S. Patent No. 5,013,556.

Fab′ fragments of the antibody of the present invention can be conjugated to the liposomes as described in Martin et al., J. Biol. Chem., 257: 286-288 (1982) via a disulfide-interchange reaction.

The novel, conformation specific monoclonal TFF antibodies and data described herein were generated using the following materials and methods. Although the methods described herein were preferably used to generate antibodies specific for TFF1, e.g., monoclonal TFF1 antibodies, one of ordinary skill in the art would recognize that such methods could be used to produce antibodies against any TFF protein, including TFF1, TFF2 and TFF3.

### Monoclonal Antibody Production

Mice were immunized by injection of native TFF1 or TFF3 proteins. Monoclonal antibodies were produced according to standard methodologies. Hybridoma clones were expanded in culture to produce large quantities of a single type of antibody developed against a conformational epitope(s). These antibodies, called monoclonal antibodies, were examined for their specificity and sensitivity by ELISA and by native western blot analysis.

### Deposit of Biological Materials

Under the terms of the Budapest Treaty on the International Recognition of the Deposit of Microorganisms for the Purpose of Patent Procedure, the hybridoma cell lines M661/7E5/1F9, and M661/7E5/2B10, which produce antibodies referred to herein as 1F9 and 2B10, were deposited on Dec. 27, 2007, with the American Type Culture Collection (ATCC) of 10801 University Boulevard, Manassas, Va. 20110-2209 USA. The ATCC Patent Depository acknowledged the receipt and viability of the deposited cell lines on Feb. 14, 2008. The hybridoma cell line M661/7E5/2B10 was assigned ATCC Patent Deposit Designation PTA-8892. The hybridoma cell line M661/7E5/1F9 was assigned ATCC Patent Deposit Designation PTA-8893.

MTT (3-(4,5-dimethylthiazol-2-yl)-2,5-diphenyl tetrazolium bromide) Assay

A rapid colorimetric assay, based on the tetrazolium salt MTT (3-(4,5-dimethylthiazol-2-yl)-2,5-diphenyl tetrazolium bromide), that measures only living cells and can be read on a scanning multiwell spectrophotometer (ELISA reader). MTT is converted to purple formazan only when mitochondrial reductase enzymes are active, and thus conversion is directly related to the number of viable cells. The production of formazan in cells treated with an agent is measured relative to the production in control cells, and a dose-response curve can be generated.

Various cancer cell lines were purchased from the ATCC and seeded at a density of 2500 cells/100 μl media in each well of a 96-well plate. Cells were treated with different concentrations of mouse monoclonal TFF antibodies (e.g., monoclonal TFF1 antibodies described herein) (final volume 100 μl). The same procedure was performed with the same concentration of mouse IgG and utilized as a control. Microplates were incubated at 37° C. in a 5% CO₂ atmosphere for 72 h. Subsequently, 20 μl of MTT (5 mg/ml) solution was added to each well of 96-well plates containing cells treated with different concentrations of antibodies. The reaction was stopped after 4 h incubation by addition of 140 μl of 0.04 mol/L HCl in isopropanol and the absorbance of each well was measured by an ELISA reader using a test wavelength of 490 nm. Each concentration treatment was done in triplicate wells.

In Silico Conformational Epitope (CE) prediction

TFF1 and TFF3 proteins share high nucleotide sequence homology. Structures of human TFF1 and TFF3 have been resolved previously using NMR and the co-ordinates have been deposited at Protein Data Bank (PDB). NMR co-ordinates were fed into RASMOL, 3-D molecular visualization software. Homodimers formed by TFF1 and TFF3 show significant difference in the total structure and conformation.

Epitopes are defined as the portions of the antigen molecules that interact with the antigen-binding site of an antibody. Epitopes are of two types, namely, sequential epitope (SE) (when the antibody (Ab) binds to a contiguous stretch of amino acid residues that are linked by peptide bond) and conformational epitope (CE) (when Ab binds to non-contiguous residues, brought together by folding of polypeptide chain). Sequential epitopes are also referred to herein as linear epitopes. Antibodies that bind linear epitopes are Abs that bind to consecutive amino acids of an antigen sequence. Conformational epitopes are also referred to herein as native epitopes. Antibodies that bind conformational epitopes or native epitopes are Abs that bind TFF polypeptides and/or proteins in solution, e.g., physiologically compatible conditions. It is known from the analyses of the crystal structures of Ag-Ab complexes that, in order to be recognized by the antibodies, the residues must be accessible for interactions and thus be present on the surface of antigens. In addition, the conformation specific antibodies to TFF provided herein may bind to denatured TFF polypeptides or proteins or linear sequences derived from TFFs.

A publicly available algorithm was used to predict SE and CE of TFF1 and TFF3. The results are shown below in Tables 1-6.

TABLE 1

Conformational epitopes predicted
on the TFF1 homodimer.

| | | |
|---|---|---|
| CE | | Res within 6Å |
| No. | AD within 6Å of Reference AD | of Ref AD |

| | | | |
|---|---|---|---|
| 1 | A_1: | EAQTETCTVApRErQN:16 | A_43: W |
| | | (SEQ ID NO: 1) | |
| | A_19: | FPGvTPSQcANKG:31 | |
| | | (SEQ ID NO: 2) | |
| | A_34: | FDDTVRG:40 | |
| | | (SEQ ID NO: 3) | |
| | A_46: | YPnTIDVPPEEECEF:60 | |
| | | (SEQ ID NO: 4) | |
| | B_46: | YPNTIDVPPEEECEF:60 | |
| | | (SEQ ID NO: 4) | |
| 2 | A_19: | FPGvTPSQcANKG:31 | A_43: W |
| | | (SEQ ID NO: 2) | |
| | A_1: | EAQTETCTVApRErQN:16 | |
| | | (SEQ ID NO: 1) | |
| | A_34: | FDDTVRG:40 | |
| | | (SEQ ID NO: 3) | |
| | A_46: | YPnTIDVPPEEECEF:60 | |
| | | (SEQ ID NO: 4) | |
| 3 | A_34: | FDDTVRG:40 | |
| | | (SEQ ID NO: 3) | |
| | A_1: | EAQTETCTVApRErQN:16 | |
| | | (SEQ ID NO: 1) | |
| | A_19: | FPGvTPSQcANKG:31 | |
| | | (SEQ ID NO: 2) | |
| 4 | A_46: | YPnTIDVPPEEECEF:60 | |
| | | (SEQ ID NO: 4) | |
| | A_1: | EAQTETCTVApRErQN:16 | |
| | | (SEQ ID NO: 1) | |
| | A_19: | FPGvTPSQcANKG:31 | |
| | | (SEQ ID NO: 2) | |
| | A_46: | YPNTIDVPPEEECEF:60 | |
| | | (SEQ ID NO: 4) | |
| 5 | B_1: | EAQTETcTvAPRErQNcGFPGvTPSQc | |
| | | ANKG:31 | |
| | | (SEQ ID NO:5) | |
| | B_34: | FdDTVRG:40 | |
| | | (SEQ ID NO: 3) | |
| | B_46: | YPNTIDVPPEEECEF:60 | |
| | | (SEQ ID NO: 4) | |
| 6 | B_46: | FdDTVRG:40 | |
| | | (SEQ ID NO: 3) | |
| | B_1: | EAQTETcTVAPRErQNcGFPGvTPSQc | |
| | | ANKG:31 | |
| | | (SEQ ID NO: 5) | |
| 7 | B_46: | YPNTIDVPPEEECEF:60 | |
| | | (SEQ ID NO: 4) | |
| | A_1: | EAQTETCTVApRErQN:16 | |
| | | (SEQ ID NO: 1) | |
| | A_46: | YPnTIDVPPEEECEF:60 | |
| | | (SEQ ID NO: 4) | |
| | B_1: | EAQTETcTvAPRErQNcGFPGvTPSQc | |
| | | ANKG:31 | |
| | | (SEQ ID NO: 5) | |

AD - antigenic determinants; CE - conformational epitope; A and B are two TFF1 proteins forming the homodimer.

Table 1 shows the seven different conformational epitopes present in TFF1 homodimer. Any AD found within 6 ø distance to the reference AD forms a part of a conformational epitope. There were no sequential epitopes (SE) predicted. Upper case shows the amino-acids available at the surface and lower case shows the amino acids embedded within the tertiary structure and having less than 25% accessibility to the antibody.

TABLE 2

Antigenic determinants predicted on
TFF1 homodimer.
PREDICTED AD

| | |
|---|---|
| AD No. | Antigenic Determinant |

| | | |
|---|---|---|
| 1_A | A_1: | EAQTETCTVApRErQN:16 |
| | | (SEQ ID NO: 1) |
| 2_A | A_19: | FPGvTPSQcANKG:31 |
| | | (SEQ ID NO: 2) |
| 3_A | A_34: | FDDTVRG:40 |
| | | (SEQ ID NO: 3) |
| 4_A | A_46: | YPnTIDVPPEEECEF:60 |
| | | (SEQ ID NO: 4) |
| 5_B | B_1: | EAQTETcTvAPRErQNcGFPGvTPSQcANKG:31 |
| | | (SEQ ID NO: 5) |
| 6_B | B_34: | FdDTVRG:40 |
| | | (SEQ ID NO: 3) |
| 7_B | B_46: | YPNTIDVPPEEECEF:60 |
| | | (SEQ ID NO: 4) |

AD - antigenic determinants; A and B are two TFF1 proteins forming the homodimer.

Table 2 shows the seven different antigen determinants (AD) found in the native TFF1 homodimer. The AD is from 7 to 31 amino acids in length spanning more than 80% of the primary protein sequence is involved in forming different conformational epitopes.

TABLE 3
Conformational epitopes predicted on TFF1 monomer.

| | | |
|---|---|---|
| CE | Predicted CE formed by AD | Res within 6Å |
| No. | within 6Å of Reference AD | of Ref AD |

| | | | |
|---|---|---|---|
| 1 | A_1: | EAQTETCTVApRErQN:16 | A_43: W |
| | | (SEQ ID NO: 1) | |
| | A_19: | FPGvTPSQcANKG:31 | |
| | | (SEQ ID NO: 2) | |
| | A_34: | FDDTVRG:40 | |
| | | (SEQ ID NO: 3) | |
| | A_46: | YPnTIDVPPEEECEF:60 | |
| | | (SEQ ID NO: 4) | |
| 2 | A_34: | FDDTVRG:40 | A_43: W |
| | | (SEQ ID NO: 3) | |
| | A_1: | EAQTETCTVApRErQN:16 | |
| | | (SEQ ID NO: 1) | |
| | A_19: | FPGvTPSQcANKG:31 | |
| | | (SEQ ID NO: 2) | |
| 3 | A_46: | YPnTIDVPPEEECEF:60 | |
| | | (SEQ ID NO: 4) | |
| | A_1: | EAQTETCTVApRErQN:16 | |
| | | (SEQ ID NO: 1) | |
| | A_19: | FPGvTPSQcANKG:31 | |
| | | (SEQ ID NO: 2) | |

AD - antigenic determinants; CE - conformational epitope.

Table 3 shows the three different conformational epitopes present in TFF1 monomer. Any AD found within 6 ø distance to the reference AD forms a part of a conformational epitope. There were no sequence epitopes found. Upper case shows the amino-acids available at the surface and lower case shows the amino acids embedded within the tertiary structure and having less than 25% accessibility to the antibody.

TABLE 4

Antigenic determinants predicted on TFF1 monomer.
PREDICTED AD

| | |
|---|---|
| AD No. | Antigenic Determinant |

| | | |
|---|---|---|
| 1_A | A_1: | EAQTETCTVApRErQN-16 |
| | | (SEQ ID NO: 1) |
| 2_A | A_19: | FPGvTPSQcANKG:31 |
| | | (SEQ ID NO: 2) |
| 3_A | A_34: | FDDTVRG-40 |
| | | (SEQ ID NO: 3) |
| 4_A | A_46: | YPnTIDVPPEEECEF-60 |
| | | (SEQ ID NO: 4) |

AD - antigenic determinants.

Table 4 shows the four different antigen determinants (AD) found in the TFF1 homodimer. The AD is from 7 to 16 amino acids in length spanning more than 80% of the primary protein sequence is involved in forming different conformational epitopes.

TABLE 5

Conformational epitopes predicted on
TFF3 homodimer.

| | | |
|---|---|---|
| CE | | Res within 6Å |
| No. | AD within 6Å of Reference AD | of Ref AD |

| | | | |
|---|---|---|---|
| 1 | A_1: | EEYVTLsAN:9 | A_52: L |
| | | (SEQ ID NO: 6) | |
| | A_12: | AvPaKDrVDcGyPHvTPKEcNN:33 | A_59: F |
| | | (SEQ ID NO: 7) | |
| | B_1: | EEYVGLsAN:9 | |
| | | (SEQ ID NO: 8) | |
| 2 | A_12: | AvPaKDrVDcGyPHvTPKEcNN:33 | A_43: W |
| | | (SEQ ID NO: 7) | |
| | A_1: | EEYVGLsAN:9 | |
| | | (SEQ ID NO: 8) | |
| | A_40: | SRIPG:44 | |
| | | (SEQ ID NO: 9) | |
| | B_12: | AvPaKDrVDcGyPHVTPKEcNN:33 | |
| | | (SEQ ID NO: 7) | |
| 3 | A_40: | SRIPG:40 | A_43: W |
| | | (SEQ ID NO: 9) | |
| | A_12: | AvPaKDrVDcGyPHvTPKEcNN:33 | |
| | | (SEQ ID NO: 7) | |
| 4 | B_1: | EEYVGLsAN:9 | |
| | | (SEQ ID NO: 8) | |
| | B_1: | EEYVGLsAN:9 | |
| | | (SEQ ID NO: 8) | |
| | B_12: | AvPaKDrVDcGyPHVTPKEcNN:33 | |
| | | (SEQ ID NO: 7) | |
| 5 | B_12: | AvPaKDrVDcGyPHVTPKEcNN:33 | |
| | | (SEQ ID NO: 7) | |
| | A_12: | AvPaKDrVDcGyPHvTPKEcNN:33 | |
| | | (SEQ ID NO: 7) | |
| | B_38: | FdSRIPG:44 | |
| | | (SEQ ID NO: 10) | |
| | B_1: | EEYVGLsAN:9 | |
| | | (SEQ ID NO: 8) | |
| 6 | B_38: | FdSRIPG:44 | |
| | | (SEQ ID NO: 10) | |
| | B_12: | AvPaKDrVDcGyPHVTPKEcNN:33 | |
| | | (SEQ ID NO: 7) | |

AD - antigenic determinants; CE - conformational epitope; A and B are two TFF3 proteins forming the homodimer.

Table 5 shows the six different conformational epitopes present in TFF3 homodimer. Any AD found within 6A distance to the reference AD forms a part of a conformational epitope. Similar to TFF1 homodimer there were no sequential epitope (SE) found. None of the CE was found overlapping with TFF1 homodimer. Upper case shows the amino-acids available at the surface and lower case shows the amino acids embedded within the tertiary structure and having less than 25% accessibility to the antibody.

TABLE 6

Antigenic determinants predicted
on TFF3 homodimer.
PREDICTED AD

| | |
|---|---|
| AD No. | Antigenic Determinant |

| | | |
|---|---|---|
| 1_A | A_1: | EEYVTLsAN:9 |
| | | (SEQ ID NO: 6) |
| 2_A | A_12: | AvPaKDrVDcGyPHvTPKEcNN:33 |
| | | (SEQ ID NO: 7) |
| 3_A | A_40: | SRIPG:44 |
| | | (SEQ ID NO: 9) |
| 4_A | B_1: | EEYVGLsAN:9 |
| | | (SEQ ID NO: 8) |
| 5_A | B_12: | AvPaKDrVDcGyPHVTPKEcNN:33 |
| | | (SEQ ID NO: 7) |
| 6_A | B_38: | FdSRIPG:44 |
| | | (SEQ ID NO: 10) |

AD - antigenic determinants; A and B are the two TFF3 molecules forming homodimer.

Table 6 shows the six different antigen determinants (AD) found in the native TFF1 homodimer. The AD is from 5 to 21 amino acids in length spanning 50% of the primary sequence of the protein involved in forming different conformational epitopes.

The monomeric and dimeric forms of the native TFF1 and dimeric form of TFF3 NMR resolved 3D structures were used to predict their epitopes. Both native TFF1 and TFF3 have multiple conformational epitopes (CE) spanning the exposed native surface structure and do not posses any sequential epitopes (SE). The two antigens exhibit different CE's concordant with the antigen specificity of the antibodies observed in native western. TFF1 monomer and dimer share two epitopes in common. TFF1 and TFF3 do not share any antigenic determinants (AD) in common.

Diagnosis of Cancers and/or Proliferation Disorders

The conformation specific antibodies to TFF described herein are useful in a variety of diagnostic applications. The invention features a method for diagnosing cancer or a cell proliferation and/or survival disorder in a mammal by contacting a tissue or bodily fluid from the mammal with a conformation specific antibody to TFF under conditions sufficient to form an antigen-antibody complex and detecting the antigen-antibody complex. Cancers or tumors detected using the conformation specific antibodies to TFF described herein include an epithelial tumor such as, e.g., lung cancer, colorectal cancer, breast cancer, pancreatic cancer, ovarian cancer, prostate cancer, hepatic carcinoma, gastric carcinoma, endometrial carcinoma, renal carcinoma, thyroid cancer, biliary duct cancer, esophageal cancer, brain cancer, melanoma, multiple myeloma, hematologic tumor, and lymphoid tumor. Proliferative disorders detected using the conformation specific antibodies to TFF described herein include, e.g., keratinocyte hyperproliferation, inflammatory cell infiltration, cytokine alteration, endometriosis, epidermic and dermoid cysts, lipomas, adenomas, capillary and cutaneous hemangiomas, lymphangiomas, nevi lesions, teratomas, nephromas, myofibromatosis, osteoplastic tumors, and other dysplastic masses.

Methods for diagnosis include detecting a tumor cell in vivo or ex vivo in bodily fluids or in tissue. For example, a biopsied tissue is contacted with an antibody and antibody binding measured. In addition to biopsied tissue samples, whole blood, serum, plasma, stool, urine, cerebrospinal fluid, bronchoalveolar lavage, sputum, exhaled breath condensate, semen, saliva, joint fluid or ulcer secrete is tested. Whole body diagnostic imaging may be carried out to detect microtumors undetectable using conventional diagnostic methods. Accordingly, a method for diagnosing a tumor in a mammal is carried out by contacting a tissue, e.g., a lymph node, of a mammal with a detectably-labeled antibody which binds to a TFF conformational epitope. An increase in the level of antibody binding at a tissue site compared to the level of binding to a normal non-neoplastic tissue indicates the presence of a neoplasm at the tissue site. For detection purposes, the antibody is labeled with a detectable marker, e.g., non-radioactive tag, a radioactive compound, or a colorimetric agent. For example, the antibody or antibody fragment is tagged with ¹²⁵I, ⁹⁹Tc, Gd⁺⁺⁺, or Fe⁺⁺. Green fluorescent protein is used as a colorimetric tag.

A method for diagnosis or prognosis is carried out by contacting a bodily fluid or tissue sample from the mammal with an antibody under conditions sufficient to form an antigen-antibody complex and detecting the antigen-antibody complex; quantitating the amount of complex to determine the level of TFF, and comparing the level with a normal control level of TFF. For prognostic purposes, an increasing level of TFF over time indicates a progressive worsening of the disease, and therefore, an adverse prognosis.

Patient derived tissue samples, e.g., biopsies of solid tumors, as well as bodily fluids such as a CNS-derived bodily fluid, blood, serum, urine, saliva, sputum, lung effusion, and ascites fluid, are contacted with a conformation specific antibodies to TFF.

Detecting an increase in TFF or TFF gene products in a patient-derived tissue sample (e.g., solid tissue or bodily fluid) is carried out using standard methods, e.g., by Western blot assays or a quantitative assay such as ELISA. For example, a standard competitive ELISA format using a conformation specific antibody to TFF is used to quantify patient TFF levels. Alternatively, a sandwich ELISA using a first antibody as the capture antibody and a second conformation specific antibody as a detection antibody is used.

Methods of detecting TFF include contacting a component of a bodily fluid with a conformation specific antibody bound to solid matrix, e.g., microtiter plate, bead, dipstick. For example, the solid matrix is dipped into a patient-derived sample of a bodily fluid, washed, and the solid matrix is contacted with a reagent to detect the presence of immune complexes present on the solid matrix.

Proteins in a test sample are immobilized on (e.g., bound to) a solid matrix. Methods and means for covalently or non-covalently binding proteins to solid matrices are known in the art. The nature of the solid surface may vary depending upon the assay format. For assays carried out in microtiter wells, the solid surface is the wall of the microtiter well or cup. For assays using beads, the solid surface is the surface of the bead. In assays using a dipstick (i.e., a solid body made from a porous or fibrous material such as fabric or paper) the surface is the surface of the material from which the dipstick is made. Examples of useful solid supports include nitrocellulose (e.g., in membrane or microtiter well form), polyvinyl chloride (e.g., in sheets or microtiter wells), polystyrene latex (e.g., in beads or microtiter plates, polyvinylidine fluoride (known as IMMULON™), diazotized paper, nylon membranes, activated beads, and Protein A beads. The solid support containing the antibody is typically washed after contacting it with the test sample, and prior to detection of bound immune complexes. Incubation of the antibody with the test sample is followed by detection of immune complexes by a detectable label. For example, the label is enzymatic, fluorescent, chemiluminescent, radioactive, or a dye. Assays which amplify the signals from the immune complex are also known in the art, e.g., assays which utilize biotin and avidin.

A TFF-detection reagent, e.g., a conformation specific antibody, is packaged in the form of a kit, which contains one or more conformation specific antibodies, control formulations (positive and/or negative), and/or a detectable label. The assay may be in the form of a standard two-antibody sandwich assay format known in the art.

### Administration of Compositions for Cancer Therapy

The conformation specific TFF-specific antibodies described herein are used to inhibit the growth of a tumor cell, or kill the tumor cell. In addition to cancer therapy, the methods are useful to confer clinical benefit to those suffering from or at risk of developing a precancerous condition or lesion or a non-cancerous hyperproliferative disorder.

The agent (e.g., peptides or nucleic acids of the invention) of use in inhibiting a TFF may be used on their own, or in the form of compositions in combination with one or more pharmaceutically acceptable diluents, carriers and/or excipients.

As-used herein, the phrase “pharmaceutically acceptable diluents, carriers and/or excipients” is intended to include substances that are useful in preparing a pharmaceutical composition, may be co-administered with an agent in accordance with the invention while allowing same to perform its intended function, and are generally safe, non-toxic and neither biologically nor otherwise undesirable. Examples of pharmaceutically acceptable diluents, carriers and/or excipients include solutions, solvents, dispersion media, delay agents, emulsions and the like. Diluents, carriers and/or excipients may contain minor amounts of additives such as substances that enhance isotonicity and chemical stability.

A variety of pharmaceutically acceptable diluents, carriers and/or excipients known in the art may be employed in compositions of the invention. As will be appreciated, the choice of such diluents, carriers and/or excipients will be dictated to some extent by the nature of the agent to be used, the intended dosage form of the composition, and the mode of administration thereof. By way of example, in the case of administration of nucleic acids such as vectors adapted to express antisense or iRNA, suitable carriers include isotonic solutions, water, aqueous saline solution, aqueous dextrose solution, and the like.

In addition to standard diluents, carriers and/or excipients, a pharmaceutical composition of the invention may be formulated with additional constituents, or in such a manner, so as to enhance the activity of the agent or help protect the integrity of the agent. For example, the composition may further comprise adjuvants or constituents which provide protection against degradation, or decrease antigenicity of an agent, upon administration to a subject. Alternatively, the agent may be modified so as to allow for targeting to specific cells, tissues or tumors.

Additionally, the antibodies are formulated with other ingredients which may be of benefit to a subject in particular instances. For example, optionally, one or more anti-neoplastic agents are co-administered or incorporated into the formulation. Examples of such agents include: alkylating agents (e.g., chlorambucil (Leukeran™), cyclophosphamide (Endoxan™, Cycloblastin™, Neosar™, Cyclophosphamide™), ifosfamide (Holoxan™, Ifex™, Mesnex™), thiotepa (Thioplex™, Thiotepa™)); antimetabolites/S-phase inhibitors (e.g., methotrexate sodium (Folex™, Abitrexate™, Edertrexate™), 5-fluorouracil (Efudix™, Efudex™), hydroxyurea (Droxia™, Hydroxyurea, Hydrea™), amsacrine, gemcitabine (Gemzar™), dacarbazine, thioguanine (Lanvis™)); antimetabolites/mitotic poisons (e.g., etoposide (Etopophos™, Etoposide, Toposar™), vinblastine (Velbe™, Velban™), vindestine (Eldesine™), vinorelbine (Navelbine™), paclitaxel (Taxol™)); antibiotic-type agents (e.g., doxorubicin (Rubex™), bleomycin (Blenoxane™), dactinomycin (Cosmegen™), daunorubicin (Cerubidin™), mitomycin (Mutamycin™)); hormonal agents (e.g., aminoglutethimide (Cytadren™); anastrozole (Arimidex™), estramustine (Estracyt™, Emcyt™), goserelin (Zoladex™), hexamethylmelanine (Hexamet™), letrozole (Femara™), anastrozole (Arimidex™), tamoxifen (Estroxyn™, Genox™, Novaldex™, Soltamox™, Tamofen™)); or any combination of any two or more anti-neoplastic agents (e.g., Adriamycin/5-fluorouracil/cyclophosphamide (FAC), cyclophosphamide/methotrexate/5-fluorouracil (CMF)). The antibodies of the invention may also be formulated with compounds and agents, other than those specifically mentioned herein, in accordance with accepted pharmaceutical practice.

In accordance with the mode of administration to be used, and the suitable pharmaceutical excipients, diluents and/or carriers mentioned herein before, compositions of the invention are converted to customary dosage forms such as solutions, orally administrable liquids, injectable liquids, tablets, coated tablets, capsules, pills, granules, suppositories, transdermal patches, suspensions, emulsions, sustained release formulations, gels, aerosols, liposomes, powders and immunoliposomes. The dosage form chosen will reflect the mode of administration desired to be used, the disorder to be treated and the nature of the agent to be used. Particularly preferred dosage forms include orally administrable tablets, gels, pills, capsules, semisolids, powders, sustained release formulation, suspensions, elixirs, aerosols, ointments or solutions for topical administration, and injectable liquids.

Skilled persons will readily recognize appropriate dosage forms and formulation methods. The compositions can be prepared by contacting or mixing specific agents and ingredients with one another. Then, if necessary, the product is shaped into the desired formulation. By way of example, certain methods of formulating compositions may be found in references such as Gennaro AR: Remington: The Science and Practice of Pharmacy, 20th ed., Lippincott, Williams & Wilkins, 2000.

The amount of an antibody of the invention in a composition can vary widely depending on the type of composition, size of a unit dosage, kind of carriers, diluents and/or excipients, and other factors well known to those of ordinary skill in the art. The final composition can comprise from 0.0001 percent by weight (% w) to 100% w of the actives of this invention, preferably 0.001% w to 10% w, with the remainder being any other active agents present and/or carrier(s), diluent(s) and/or excipient(s).

Administration of any of the agents or compositions of the invention can be by any means capable of delivering the desired activity (inhibition of tumor cell proliferation) to a target site within the body of a subject. A “target site” may be any site within the body which may have or be susceptible to a proliferative disorder, and may include one or more cells, tissues or a specific tumor.

For example, administration may include parenteral administration routes, systemic administration routes, oral and topical administration. For example, administration may be by way of injection, subcutaneous, intraorbital, ophthalmic, intraspinal, intracisternal, topical, infusion (using e.g. slow release devices or minipumps such as osmotic pumps or skin patches), implant, aerosol, inhalation, scarification, intraperitoneal, intracapsular, intramuscular, intratumoral, intranasal, oral, buccal, transdermal, pulmonary, rectal or vaginal. As will be appreciated, the administration route chosen may be dependent on the position of the target site within the body of a subject, as well as the nature of the agent or composition being used.

The dose of an antibody of the invention or composition administered, the period of administration, and the general administration regime may differ between subjects depending on such variables as the nature of the condition to be treated, severity of symptoms of a subject, the size of any tumor to be treated, the target site to be treated, the mode of administration chosen, and the age, sex and/or general health of a subject. Persons of general skill in the art to which the invention relates will readily appreciate or be able to determine appropriate administration regimes having regard to such factors, without any undue experimentation. Administration of an antibody of the invention is in an amount necessary to at least partly attain a desired response. Administration may include a single daily dose or administration of a number of discrete divided doses as may be appropriate. Administration regimes can combine different modes or routes of administration. For example, intratumoral injection and systemic administration can be combined.

The method may further comprise further steps such as the administration of additional agents or compositions which may be beneficial to a subject having regard to the condition to be treated. For example, other agents of use in treating proliferative disorders (such as the anti-neoplastic agents mentioned above) could be administered. It should be appreciated that such additional agents and compositions may be administered concurrently with the agents and compositions of the invention, or in a sequential manner (for example the additional agents or compositions could be administered before or after administration of the agents or compositions of the invention. It should be appreciated in relation to sequential delivery of agents or compositions, that sequential administration of one agent or composition after the other need not occur immediately, although this may be preferable. There may be a time delay between delivery of the agents or compositions. The period of the delay will depend on factors such as the condition to be treated and the nature of the compositions or agents to be delivered. However, by way of example, the delay period can be between several hours to several days or months.

### Other Embodiments

While the invention has been described in conjunction with the detailed description thereof, the foregoing description is intended to illustrate and not limit the scope of the invention, which is defined by the scope of the appended claims. Other aspects, advantages, and modifications are within the scope of the following claims.

### EXAMPLES

TFF proteins (e.g., TFF1 and TFF3) are over-expressed in cancer cells of various organs and induce invasion, survival and proliferation of neoplastic cells. The studies described herein were designed to evaluate the neutralization of secreted TFF1 proteins using TFF specific antibodies (e.g., monoclonal TFF1 antibodies) as cytotoxic agents on cancer cells.

### Example 1

### Inhibition of Cell Survival in Human Gastric Carcinoma Cells by Conformation Specific Monoclonal TFF1 Antibodies

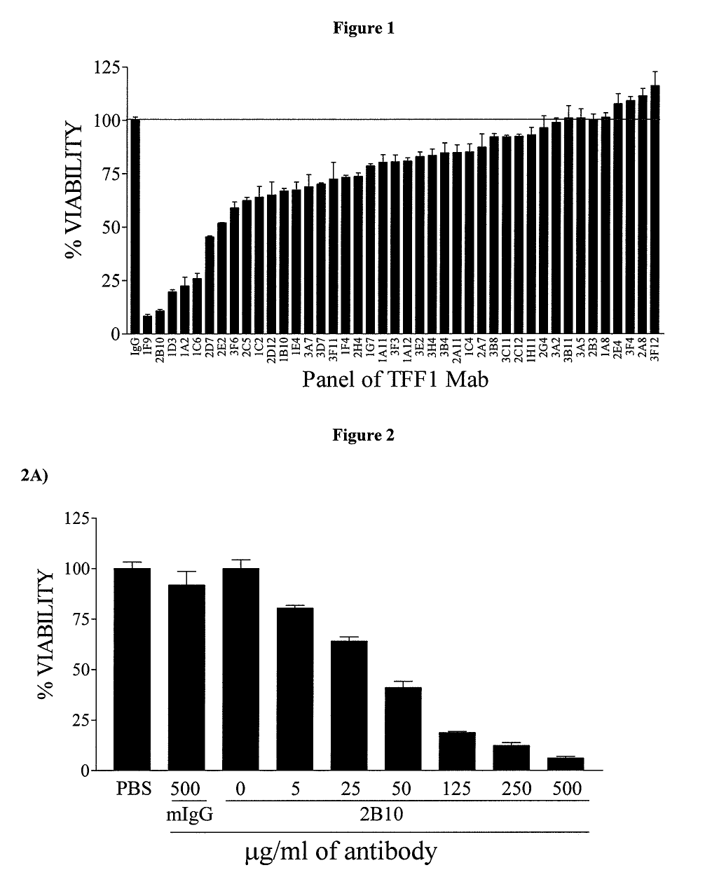

A panel of TFF1 mouse monoclonal antibodies were screened for functional efficacy against the human gastric carcinoma cell line, AGS, which expresses TFF1. Cell survival in response to treatment with these antibodies was determined by MTT assay, as described above. Each antibody was added at a final concentration of 500 ug/mL. Mouse IgG (500 ug/mL) was used as a control. The results of the assay are shown in FIG. 1. Each point represents mean a Standard Error (SE) of triplicate determinations. As shown in FIG. 1, several conformation specific monoclonal TFF1 antibodies inhibited survival of AGS, human gastric carcinoma cells. Two monoclonal antibodies, 1F9 and 2HB10, highly reduced AGS cell viability at a concentration of 500 ug/mL.

### Example 2

### Inhibition of Cell Survival in Human Gastric Carcinoma Cells by Conformation Specific Monoclonal TFF1 Antibody 2B10

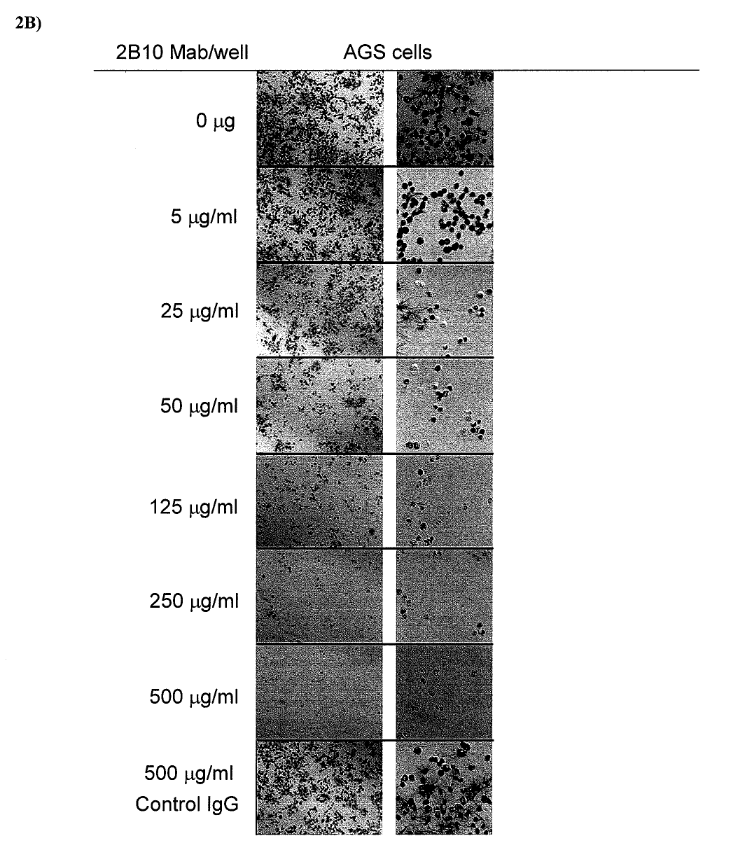

TFF1 monoclonal antibody 2B10 was screened for functional efficacy in a dose-dependent manner against AGS human gastric carcinoma cells, which express TFF1. AGS human gastric carcinoma cell survival was determined by MTT assay, as described above. TFF1 monoclonal antibody 2B10 was added at a final concentration range of 0 to 500 ug/mL (five-fold serial dilutions). Mouse IgG (500 ug/mL) was used as a control. The dose response curve is shown in FIG. 2A. Each point represents mean±Standard Error (SE) of triplicate determinations. Photomicrographs of the cells after addition of MTT are shown in FIG. 2B (left-side, low magnification; right side, high magnification).

As shown in FIGS. 2A and 2B, the titration of TFF1 monoclonal antibody 2B10 inhibited survival of AGS human gastric carcinoma cells in a concentration dependent manner.

### Example 3

### Inhibition of Cell Survival in Human Mammary Carcinoma Cell Lines by Conformation Specific Monoclonal TFF1 Antibody 2B10

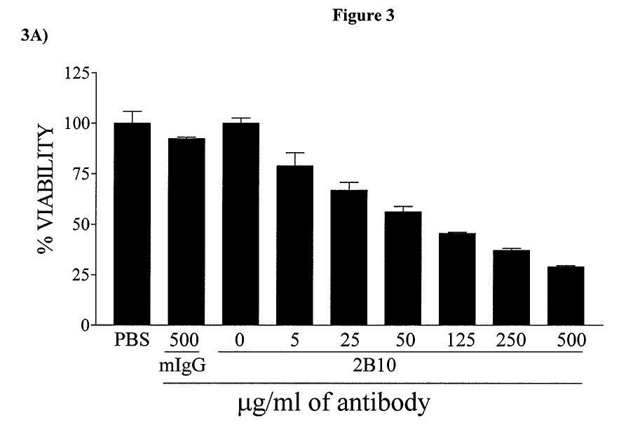

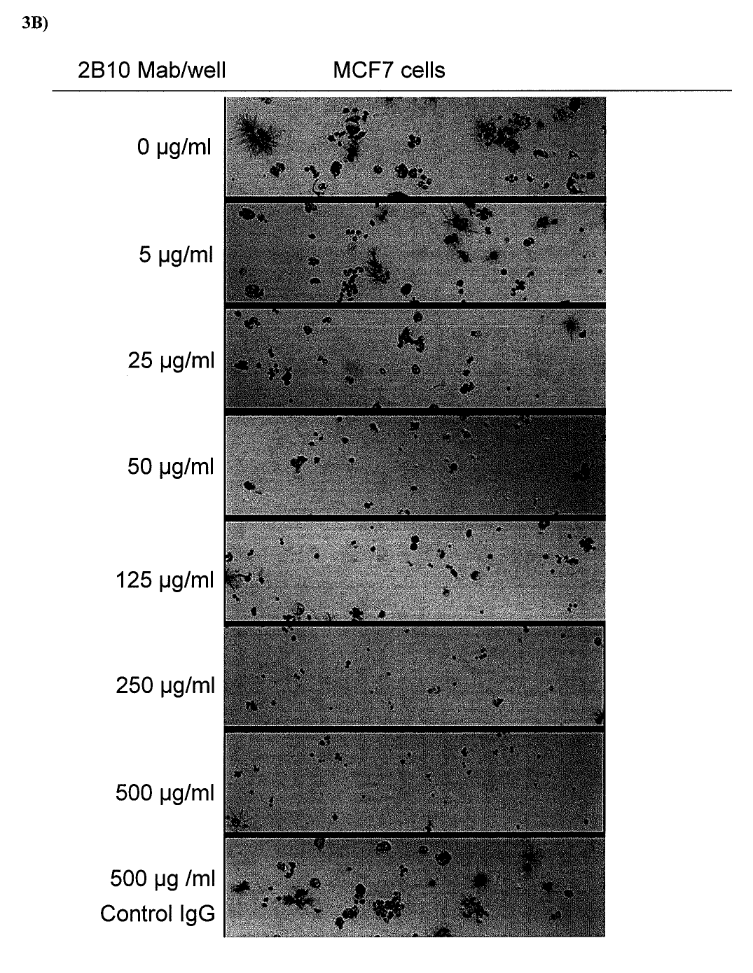

TFF1 monoclonal antibody 2B10 was screened for functional efficacy in a dose-dependent manner against MCF-7, a human mammary carcinoma cell line which expresses TFF1. MCF-7 human mammary carcinoma cell survival was determined by MTT assay, as described above. TFF1 monoclonal antibody 2B10 was added at a final concentration range of 0 to 500 ug/mL (five-fold serial dilutions). Mouse IgG (500 ug/mL) was used as a control. The dose response curve is shown in FIG. 3A. Each point represents mean±Standard Error (SE) of triplicate determinations. Photomicrographs of the cells after addition of MTT are shown in FIG. 3B.

As shown in FIGS. 3A and 3B, the titration of TFF1 monoclonal antibody 2B10 inhibited survival of MCF-7 human mammary carcinoma cells in a concentration dependent manner.

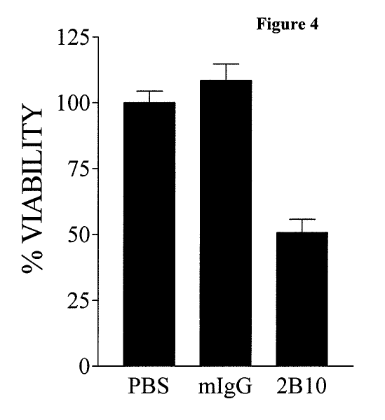

TFF1 monoclonal antibody 2B10 was also screened for functional efficacy against T47-D, a human mammary carcinoma cell line which expresses TFF1. T47-D human mammary carcinoma cell survival was determined by MTT assay, as described above. TFF1 monoclonal antibody 2B10 was added at final concentration of 500 ug/mL. Mouse IgG (500 ug/mL) was used as a control. The results of the assay are shown in FIG. 4. Each point represents mean±Standard Error (SE) of triplicate determinations. As shown in FIG. 4, the conformation specific monoclonal TFF1 antibody 2B10 inhibited survival of T47-D human mammary carcinoma cells.

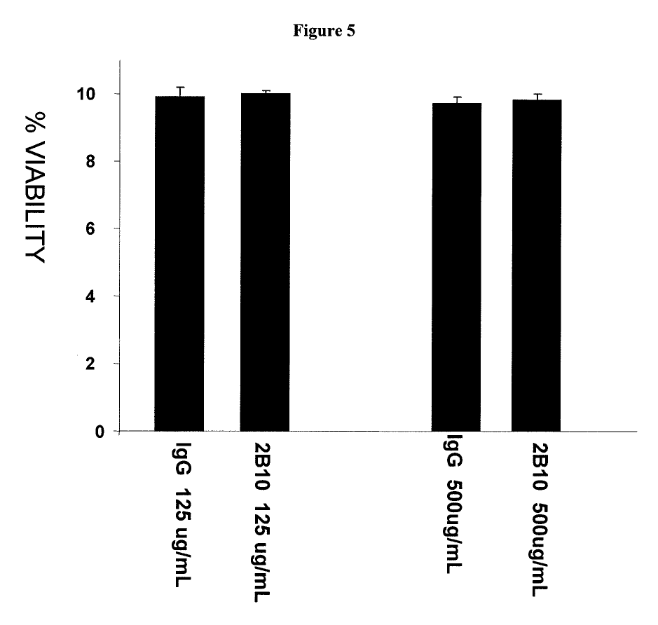

TFF1 monoclonal antibody 2B10 was also screened for functional efficacy against MDA-MB-231 human mammary carcinoma cells which lack TFF1 expression. MDA-MB-231 human mammary carcinoma cell survival was determined by MTT assay, as described above. TFF1 monoclonal antibody 2B10 was added a final concentration of either 125 ug/mL or 500 ug/mL. The results of the assay are shown in FIG. 5. Each point represents mean±Standard Error (SE) of triplicate determinations. As shown in FIG. 5, the conformation specific monoclonal TFF1 antibody 2B10 had no effect on the survival of the TFF1-negative MDA-MB-231 human mammary carcinoma cell line at either concentration tested, indicating that the TFF1 monoclonal antibody 2B10 is specific for TFF1.

### Example 4

### Inhibition of Cell Survival in Human Mammary and Gastric Carcinoma Cell Lines by Conformation Specific Monoclonal TFF1 Antibody 1F9

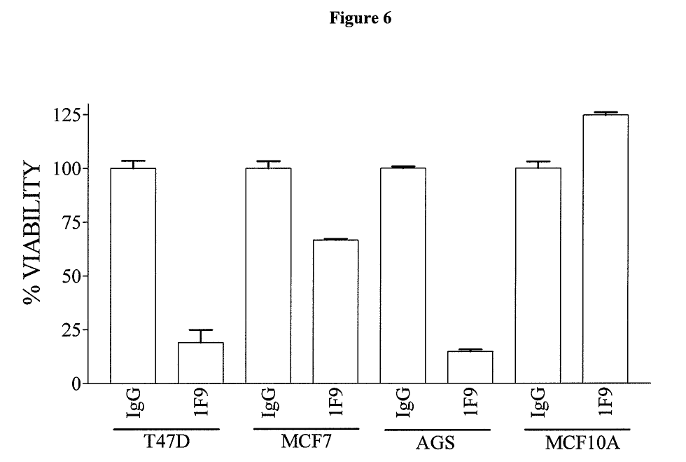

TFF1 monoclonal antibody 1F9 was screened for functional efficacy against T47-D and MCF-7 human mammary carcinoma cell lines, and AGS human gastric carcinoma cell line, each of which expresses TFF1, as well as MCF10A immortalized human mammary epithelial cells, which do not express TFF1. Cell survival was determined by MTT assay, as described above. TFF1 monoclonal antibody 1F9 was added at final concentration of 500 ug/mL. The results of the assay are shown in FIG. 6. Each point represents mean±Standard Error (SE) of triplicate determinations. As shown in FIG. 6, the conformation specific monoclonal TFF1 antibody 1F9 inhibited survival of T47-D and MCF-7 human mammary carcinoma cells and AGS human gastric carcinoma cells, but not MCF10A human epithelial cells. These results indicate the monoclonal antibody 1F9 is specific for TFF1.

While the invention has been described in conjunction with the detailed description thereof, the foregoing description is intended to illustrate and not limit the scope of the invention, which is defined by the scope of the appended claims. Other aspects, advantages, and modifications are within the scope of the following claims.

The invention will be further described in the following examples, which do not limit the scope of the invention described in the claims.

We claim:

1. An antibody that specifically binds a trefoil factor 1 (TFF1) polypeptide, wherein said antibody comprises one or both of the following characteristics:
a) said antibody binds to a conformational epitope on said TFF1 polypeptide; and
b) said antibody immunoreacts with an antigenic determinant selected from the antigenic determinants shown in Table 4.

2. The antibody of claim 1, wherein said conformational epitope is selected from a conformational epitope shown in Table 3.

3. The antibody of claim 1, wherein said antibody is selected from a multimeric antibody, a chimeric antibody composition comprising a TFF1 binding component, an antibody produced by a hybridoma cell line selected from 2B10 and 1F9, and combinations thereof.

4. An antibody that specifically binds a trefoil factor 1 (TFF1) polypeptide, wherein said antibody comprises one or both of the following characteristics:
a) said antibody binds to a conformational epitope on said TFF1 polypeptide; and
b) said antibody immunoreacts with an antigenic determinant selected from the antigenic determinants shown in Table 2.

5. The antibody of claim 4, wherein said conformational epitope is selected from a conformational epitope shown in Table 1.

6. The antibody of claim 4, wherein said antibody is selected from a multimeric antibody, a chimeric antibody composition comprising a TFF1 binding component, an antibody produced by a hybridoma cell line selected from 2B10 and 1F9, and combinations thereof.

7. The antibody of claim 1, wherein the TFF1 polypeptide is in the form of a monomer, a homodimer or a heterodimer.

8. A method of treating or preventing cancer, a cell proliferation disorder or a cell survival disorder in a subject in need thereof, comprising administering to said subject an antibody that specifically binds a trefoil factor 1 (TFF1) polypeptide, or an antibody that specifically binds a TFF1 monomer, homodimer or heterodimer polypeptide, wherein the antibody is produced by a hybridoma cell line selected from 2B10 and 1F9.

9. The method of claim 8, wherein said cancer is an epithelial cancer.

10. The method of claim 9, wherein said epithelial cancer is selected from lung cancer, colorectal cancer, breast cancer, pancreatic cancer, ovarian cancer, prostate cancer, hepatic carcinoma, gastric carcinoma, endometrial carcinoma, renal carcinoma, thyroid cancer, biliary duct cancer, esophageal cancer, brain cancer, melanoma, multiple myeloma, hematologic tumor, and lymphoid tumor.

11. The method of claim 8, wherein said cell proliferation disorder or a cell survival disorder is selected from the group consisting of keratinocyte hyperproliferation, inflammatory cell infiltration, endometriosis, cytokine alteration, epidermic and dermoid cysts, lipomas, adenomas, capillary and cutaneous hemangiomas, lymphangiomas, nevi lesions, teratomas, nephromas, myofibromatosis, osteoplastic tumors, and other dysplastic masses.

12. The method of claim 8, wherein said subject is a human.

13. The method of claim 12, further comprising the administration of a second compound wherein said second compound is a chemotherapeutic or anti-neoplastic agent.

14. A method of diagnosing a cancer, a cell proliferation disorder or a cell survival disorder in a subject, comprising
a) contacting a test sample from said subject with an antibody that specifically binds a trefoil factor 1 (TFF1) polypeptide, or an antibody that specifically binds a TFF1 monomer, homodimer or heterodimer polypeptide, wherein the antibody is produced by a hybridoma cell line selected from 2B10 and 1F9.
b) detecting the level of antibody that binds to said sample,
wherein an increase in the level of antibody binding in said sample compared to the level of binding in a control sample indicates the presence of a cancer, a cell proliferation disorder or a cell survival disorder.

15. The method of claim 14 wherein said cancer is an epithelial cancer.

16. The method of claim 15, wherein said epithelial cancer is selected from lung cancer, colorectal cancer, breast cancer, pancreatic cancer, ovarian cancer, prostate cancer, hepatic carcinoma, gastric carcinoma, endometrial carcinoma, renal carcinoma, thyroid cancer, biliary duct cancer, esophageal cancer, brain cancer, melanoma, multiple myeloma, hematologic tumor, and lymphoid tumor.

17. The method of claim 14, wherein said cell proliferation disorder or a cell survival disorder is selected from the group consisting of keratinocyte hyperproliferation, inflammatory cell infiltration, endometriosis, cytokine alteration, epidermic and dermoid cysts, lipomas, adenomas, capillary and cutaneous hemangiomas, lymphangiomas, nevi lesions, teratomas, nephromas, myofibromatosis, osteoplastic tumors, and other dysplastic masses.

18. The method of claim 14, wherein said subject is a human.


SEQUENCE LISTING (10 sequences)

SEQ ID NO: 1 - protein, 16 aa, Artificial Sequence
  chemically synthesized
EAQTETCTVAPRERQN

SEQ ID NO: 2 - protein, 13 aa, Artificial Sequence
  chemically synthesized
FPGVTPSQCANKG

SEQ ID NO: 3 - protein, 7 aa, Artificial Sequence
  chemically synthesized
FDDTVRG

SEQ ID NO: 4 - protein, 15 aa, Artificial Sequence
  chemically synthesized
YPNTIDVPPEEECEF

SEQ ID NO: 5 - protein, 31 aa, Artificial Sequence
  chemically synthesized
EAQTETCTVAPRERQNCGFPGVTPSQCANKG

SEQ ID NO: 6 - protein, 9 aa, Artificial Sequence
  chemically synthesized
EEYVTLSAN

SEQ ID NO: 7 - protein, 22 aa, Artificial Sequence
  chemically synthesized
AVPAKDRVDCGYPHVTPKECNN

SEQ ID NO: 8 - protein, 5 aa, Artificial Sequence
  chemically synthesized
SRIPG

SEQ ID NO: 9 - protein, 9 aa, Artificial Sequence
  chemically synthesized
EEYVGLSAN

SEQ ID NO: 10 - protein, 7 aa, Artificial Sequence
  chemically synthesized
FDSRIPG